(19) United States
(12) Patent Application Publication
(10) Pub. No.: US 2011/0319783 A1
(43) Pub. Date: Dec. 29, 2011

(54) GAS MIXING DEVICE FOR AN AIR-WAY MANAGEMENT SYSTEM

(75) Inventors: Claus Lindholt, Bronderslev (DK); Steen Andreassen, Aalborg (DK); Bram Wallace Smith, Te Kauwhata (NZ); Dorte Østergaard Sørensen, Vodskov (DK)

(73) Assignee: MERMAID CARE A/S, Norresundby (DK)

(21) Appl. No.: 12/666,126

(22) PCT Filed: Jun. 27, 2008
(86) PCT No.: PCT/DK08/50163

§ 371 Date: Jun. 29, 2011

(30) Foreign Application Priority Data
Jun. 29, 2007 (DK) PA 2007 00959

(51) Int. Cl. A61M 16/12; A61B 5/08

(52) U.S. Cl. 600/529; 128/203.12; 128/203.14

(57) ABSTRACT

The invention relates to a gas mixing device (10) for an air-way management system. The device has an elongated chamber (C) with a first gas inlet port (1P) being arranged for intake of atmospheric air (AA) into the chamber, the first inlet port being positioned at an end section of the internal chamber. Further, a second gas inlet port (2P) is arranged for intake of a gas (G) into the chamber (C), the second gas inlet port having an injector (INJ) from which the gas can exit into the chamber with an injection direction (ID), the injection direction having a projection (ID_proj) being oppositely directed relative to an in-flow direction (F) of the first gas inlet port (1P) so as to provide mixing of the atmospheric air and the gas. Opposite the first gas inlet port (1P) there is a breathing port (3P) for allowing an individual to breathe through the gas mixing device. The device is beneficial in that the breathing resistance is relatively low while the device simultaneously provides a sufficient gas mixing of the gasses to be mixed. Additionally, the invention provides a relatively compact gas mixing device which facilitates easy integration into e.g. a respiration mask for measurements of respiratory parameter of an individual e.g. a patient.

### FIELD OF THE INVENTION

The present invention relates to a gas mixing device for an air-way management system for an individual, the system being capable for administrating one or more gasses to the individual. The invention also relates to a tube fitting forming part of the gas mixing device, a respiration mask or a mouth piece with the gas mixing device, a respiration system with the gas mixing device, and use of the gas mixing device to determine one or more respiratory parameters of an individual.

### BACKGROUND OF THE INVENTION

Oxygen enters the body with inspiration and diffuses from the lungs into the blood. Subsequently the blood circulation transports oxygen to the tissues. Disorders of oxygen transport from the inspired air into the blood can result in a low oxygen saturation of the blood. These disorders in oxygen uptake include abnormal ventilation of the lung, seen in for example chronic obstructive pulmonary disease; abnormal oxygen diffusion in the lung, seen in for example pulmonary fibrosis; and abnormal perfusion (i.e. blood flow) through the lung. Estimation of parameters describing these oxygenation problems is important for diagnosis, monitoring and assessing appropriate therapeutic intervention. This is true in a wide variety of individuals, from those who are automatically ventilated and who often require continuous supplement of oxygen, to patients who only suffer from dyspnoe during exercise.

In clinical practice, the clinician usually relies upon simple measurements or variable estimates to assess the patient's oxygenation problems. These include qualitative estimates obtained from stethoscopy or chest X-ray. They also include more quantitative estimates such as arterial oxygen saturation, the alveolar-arterial oxygen pressure gradient, or estimates of the “effective shunt”, a parameter which to some extent describes all oxygenation problems in terms of a fraction of blood which does not flow through the lungs

In contrast to the poor clinical description of oxygenation problems, detailed experimental techniques such as the Multiple Inert Gas Elimination Technique (MIGET) have also been developed which describe the parameters of models with as many as fifty lung compartments. The parameters of these models give an accurate physiological picture of the individual. Whilst the MIGET has found widespread application as an experimental tool its use as a routine clinical tool has been somewhat limited. This is largely due to the cost and complexity of the technique.

Recently, a device and a method for determining one or more respiratory parameters relating to an individual has been disclosed in WO 00/45702 (to Andreassen et al.) for determining one or more respiratory parameters by means of the device, wherein the individual is suffering from a respiratory disorder e.g. hypoxemia. The device is controlled by a computer equipped with suitable software and includes functionality for on-line continuous data collection, automatic assessment of the timing of measurements, automatic assessment of the next target (oxygen saturation of arterial blood (SpO2)), automatic assessment of the appropriate fraction of oxygen in inspired gas (FIO2) settings to achieve the target SpO2, automatic control of the FIO2, on-line parameter estimation, and automatic assessment of the number of measurements required. The device is also known as an automatic lung parameter estimator (ALPE). WO 00/45702 regarding the ALPE-device and method is hereby by included by reference in its entirety.

In order to determine a respiratory parameter of an individual by means of the ALPE-device, it is important that appropriate and precise control of the fraction of oxygen in inspired gas (FIO2) is obtained. It is therefore necessary to vary the composition of the inspired gas i.e. mix the two or more gasses in a reproducible manner and administer the mixed gas to the individual.

U.S. Pat. No. 5,772,392 discloses a gas mixing devices for use with breathing circuit assemblies for use with breathing devices, such as respiratory therapy devices and ventilators and to methods for administering gases, such as breathable gases, like nitric oxide, in combination with other gases in a manner which facilitates the establishment of reliable delivery standards for the gases, which facilitates adequate mixing of the gases, and which reduces exposure time of the gases to one another so as to eliminate or minimize the production of toxic by products generated from such gas mixtures. The mixing device operates by an inserted wall or diaphragram in the tube with the gasses to be mixed, the wall having an aperture for permitting the gasses to be mixed to flow through the aperture so as to create turbulence and thereby mixture of the gasses. However, the aperture of the device will typically be relative narrow in order to provide sufficient turbulence, but this will result in a correspondingly high breathing resistance for an individual using the mixing device in connection with a breathing device. Furthermore, the length of the gas mixing device should be approximately 10 times the aperture diameter in order to create sufficient turbulence resulting in length of the device of 4 to 10 centimetres which makes the gas mixing device rather lengthy and not easy to integrate in e.g. a respiration mask.

Hence, an improved gas mixing device would be advantageous, and in particular a more efficient and/or reliable gas mixing device would be advantageous.

### SUMMARY OF THE INVENTION

Accordingly, the invention preferably seeks to mitigate, alleviate or eliminate one or more of the above mentioned disadvantages singly or in any combination. In particular, it may be seen as an object of the present invention to provide a gas mixing device that solves the above mentioned problems of the prior art with inter alia breathing resistance.

This object and several other objects are obtained in a first aspect of the invention by providing a gas mixing device for an air-way management system, the device comprising:
a) an elongated chamber (C),
b) a first gas inlet port (1P) being arranged for intake of atmospheric air (AA) into the chamber, the first inlet port being positioned at an end section of the internal chamber,
c) a second gas inlet port (2P) arranged for intake of a gas (G) into the chamber (C), the second gas inlet port comprising an injector (INJ) from which the gas can exit into the chamber with an injection direction (ID), the injection direction having a projection (ID_proj) being oppositely directed relative to an in-flow direction (F) of the first gas inlet port (1P) so as to provide mixing of the atmospheric air and the gas, and
d) a breathing port (3P) for allowing an individual to breathe through the gas mixing device, the breathing port being positioned opposite the first gas inlet port (1P) within the chamber (C).

The invention is particularly, but not exclusively, advantageous for obtaining a gas mixing device with a relatively low breathing resistance while simultaneously providing a sufficient gas mixing of the gasses to be mixed. Additionally, the invention provides a relatively compact gas mixing device because the projection (ID_proj) being oppositely directed relative to an in-flow direction (F) of the first gas inlet port (1P) i.e. an up-stream gas mixing provides an efficient mixing on comparably short distance relative to the known method in the fields, e.g. U.S. Pat. No. 5,772,392. This compactness of the gas mixing device of the present invention provides in particular for integration directly into or near by a respiration mask or a mouth piece to be used by an individual, which is a significant advantage for easy use by an individual.

In a preferred embodiment, the device may further comprise a deflector membrane positioned between the first inlet port (1P) and the injector (INJ). The deflector can enhance mixing of the air with the gas. Typically, the distance between the deflector membrane (DEF) and the exit of the injector (INJ) may be at least 2 mm, preferably at least 4, or more preferably at least 6 mm.

In one embodiment, the injector exit diameter may be maximum 3 mm, preferably maximum 1.5 mm, or more preferably maximum 0.5 mm. Depending on the fluid dynamic condition of the specific case, this distance can be varied as will be readily appreciated by the skilled person.

In another embodiment, the device may further comprise a gas sensor (GS) arranged for measuring a gas property resulting from the mixing of the atmospheric air and the gas. Typically, gas composition is measured and for instance an oxygen sensor can be inserted in the device. Also a carbon dioxide sensor can be inserted.

Additionally or alternatively, the device may further comprise a gas flow sensor (FS), preferably a bi-directional flow sensor, in order to assess the flow of air and/or gas into the device and possibly assess the out-going flow of gas from the device, i.e. the expired flow of gas by the individual. In order to measure more efficiently, the device may comprise a Venturi-contraction where the flow sensor is positioned.

In one embodiment, the device may comprise an air-way filter (BF) positioned between the injector (INJ) and the breathing port (3P) to ensure hygienic conditions and/or to protect any sensors within the device.

In another embodiment, the breathing port (3P) may be adapted to receive a face mask or a mouth piece to be used by an individual.

In a particular embodiment, the total internal volume of the gas mixing device i.e. the available volume for gas and air may be maximum 10 cm³, preferably maximum 15 cm³, or most preferably maximum 20 cm³. Thus, the device has a relatively low dead space compared to other gas mixing devices.

In a preferred embodiment, the gas mixing device may be designed so that the breathing resistance is maximum 0.2 Pa*s/L, preferably maximum 0.4 Pa*s/L, or most preferably maximum 0.6 Pa*s/L, at a flow through the device of approximately 50 L/min. These values of breathing resistance are significantly lower than other gas mixing devices available hitherto.

In a second aspect, the invention relates to a tube fitting forming part of the gas mixing device according to claim 1, the tube fitting comprises at least part of the elongated chamber (C), the second inlet port (2P) comprising the injector (INJ), the first inlet port (1P) and the breathing port (3P), the tubular fitting being adapted to receive:
optionally, the gas sensor (GS), and optionally
the flow sensor (FS).

In one embodiment, the tube fitting may be adapted to provide a tactile and/or audio response to a user upon correct assembly with a receiving part (RP) of the gas mixing device. Thus, the user may hear a “click-on” sound upon correct assembly. This could also be achieved by an electrical or optical sensor, using some electronics to check correct assembly and give the user a feedback depending upon correct and/or incorrect assembly. Possibly, electronics could be provided to block device operation if not correctly assembled.

In another embodiment, the tube fitting may be disposable after a single use to ensure hygienic conditions. Even further, the tube fitting may be arranged so as to allow for single use only by e.g. mechanically and/or possibly electronically locking mechanisms.

In a third aspect, the present invention relates to a respiration mask or a mouth piece comprising a gas mixing device according to the first aspect of the invention.

In a fourth aspect, the invention relates to an air-way management system for measurement of one or more respiratory parameters of an individual, the system comprising:
a respiration mask or a mouth piece according to the third aspect,
a gas supply for supplying the second inlet port with the gas, and
a control unit interconnected with the gas supply,
   wherein the control unit is arranged to receive output signals from optionally the gas sensor (GS) and optionally the flow sensor (FS), and control the gas supply in response to said output signals.

In a fifth aspect, the invention relates to use of a gas mixing device according to the first aspect for measurement of one or more respiratory parameters of an individual.

Hence, in its broadest aspect, the invention relates to a device for determining one or more respiratory parameters relating to an individual. By the term “individual” is herein understood an individual selected from the group comprising humans as well as farm animals, domestic animals, pet animals and animals used for experiments such as monkeys, rats, rabbits, etc.

By the term “respiratory parameters” is herein understood parameters relating to oxygen transport from the lungs to the blood, such as parameters related to abnormal ventilation, resistance to oxygen uptake from the lungs to the lung capillary blood, and parameters related to shunting of venous blood to the arterial blood stream. These respiratory parameters may be given as absolute values or relative values as compared to a set of standard values and the parameters may further be normalised or generalised to obtain parameters that are comparable to similar parameters measured for other individuals, at least for individuals of the same species.

### Glossary

FIO2 Fraction of oxygen in inspired gas.
PIO2 Pressure of oxygen in inspired gas.
SaO2 Oxygen saturation of arterial blood, measured from a blood sample.
PaO2 Pressure of oxygen in arterial blood, measured from a blood sample.
SpO2 Oxygen saturation of arterial blood, measured transcutaneously.
PpO2 Pressure of oxygen in arterial blood, measured transcutaneously.
FĒCO2 Fraction of carbon dioxide in the mixed expired gas.
FE′O2 Fraction of oxygen in expired gas at the end of expiration.
FĒO2 Fraction of oxygen in the mixed expired gas.
PĒCO2 Pressure of oxygen in the mixed expired gas.
PE′O2 Pressure of oxygen in expired gas at the end of expiration.
Vt Tidal volume, i.e. volume of gas breathed per breath.
f Respiratory frequency, i.e. number of breaths per minute.
VO2 Oxygen consumption, i.e. the amount of oxygen consumed by the tissues per minute.
Vd Dead space i.e. the volume of the lung not involved in exchanging gases with the blood.
shunt Respiratory parameter representing the faction of blood not involved in gas exchange.
Rdiff Respiratory parameter representing a resistance to oxygen diffusion across the alveolar lung capillary membrane.
\dot{V} Ventilation.
\dot{V}/\dot{Q} Respiratory parameter representing the balance between ventilation and perfusion in a region of the lung.
V-shift Respiratory parameter representing a vertical shift in plots of FIO2 against SaO2, FIO2 against SpO2, FE′O2 against SaO2, or FE′O2 against SpO2 .
H-shift Respiratory parameter representing a horizontal shift in plots of FIO2 against SaO2, FIO2 against SpO2, FE′O2 against SaO2, or FE′O2 against SpO2.

The first, second, third, fourth, and fifth aspect of the present invention may each be combined with any of the other aspects. These and other aspects of the invention will be apparent from and elucidated with reference to the embodiments described hereinafter.

### BRIEF DESCRIPTION OF THE FIGURES

The present invention will now be explained, by way of example only, with reference to the accompanying Figures, where

FIG. 1 is a schematic drawing of air-way management system according to the present invention,

FIG. 2 is a sketch of how an individual can wear a respiration mask including the gas mixing device according to the present invention,

FIG. 3 is a schematic drawing of a gas mixing device according to the present invention,

FIG. 4 is a more detailed embodiment of a gas mixing device according to the present invention,

FIG. 5 is a drawing of how the in-flow trough the gas mixing device is orientated relative to the injection direction,

FIG. 6 is a perspective view of an embodiment of the gas mixing device according to the present invention,

FIG. 7 (AA and TOP) are cross-sectional and top views, respectively, of the gas mixing device of FIG. 6,

FIG. 8 is an exploded view of the of the gas mixing device of FIG. 6, and

FIG. 9 is a graph showing oxygen saturation (SaO2) versus end tidal fraction of inspired oxygen (FE′O2) that can be beneficially measured using an air-way management system according to the present invention.

### DETAILED DESCRIPTION OF AN EMBODIMENT

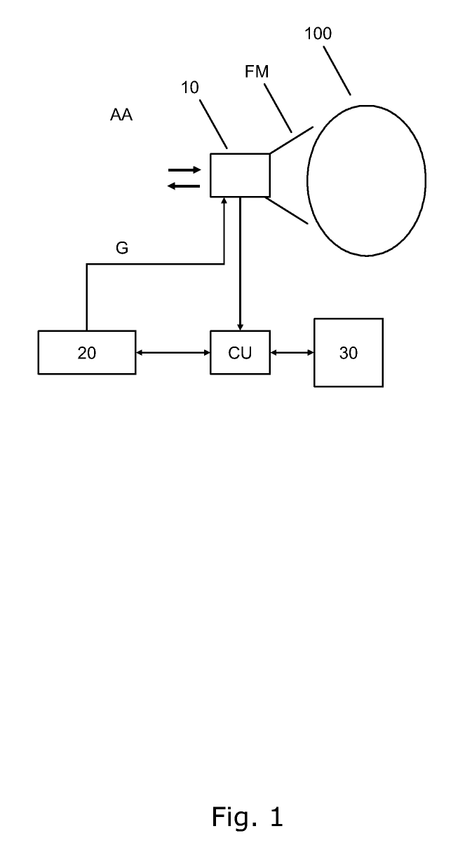

FIG. 1 is a schematic drawing of air-way management system according to the present invention. The air-way management system comprise a gas supply 20 connected to the gas mixing device 10 for supplying the gas G thereto. The gas mixing device 10 allows the individual 100, e.g. a patient to be subjected for respiratory diagnosis, to breathe through the face mask FM mounted on the gas mixing device 10 as indicated by the double arrows to the left of the gas mixing device 10. The mask FM has be air tight and alternatively a mouth piece with a nasal clamps can be used. The face mask FM and the gas mixing device 10 constitute a respiration mask in combination. The breathing takes places through a first inlet port (not shown in FIG. 1) which is in air contact with the surrounding atmosphere AA.

The gas supply 20 is operably connected to a control unit CU adapted to control the supply 20 in order to measure of one or more respiratory parameters of the individual 100. Within the device 10 a gas sensor GS, typically an oxygen sensor, and a flow sensor FS are situated (neither shown in FIG. 1). The gas sensor and the flow sensor give appropriate output signals indicative of one or more gas properties, e.g. composition, and the flow through the gas mixing device, the control unit CU being arranged to receive said output signals from the gas sensor GS and the flow sensor FS, and control the gas supply 20 in response to said output signals. The control unit CU is operably connected to a display device 30 to communicate to a user, e.g. a nurse or a physician, information related to the air-way management system and, possibly, one or more respiratory parameters.

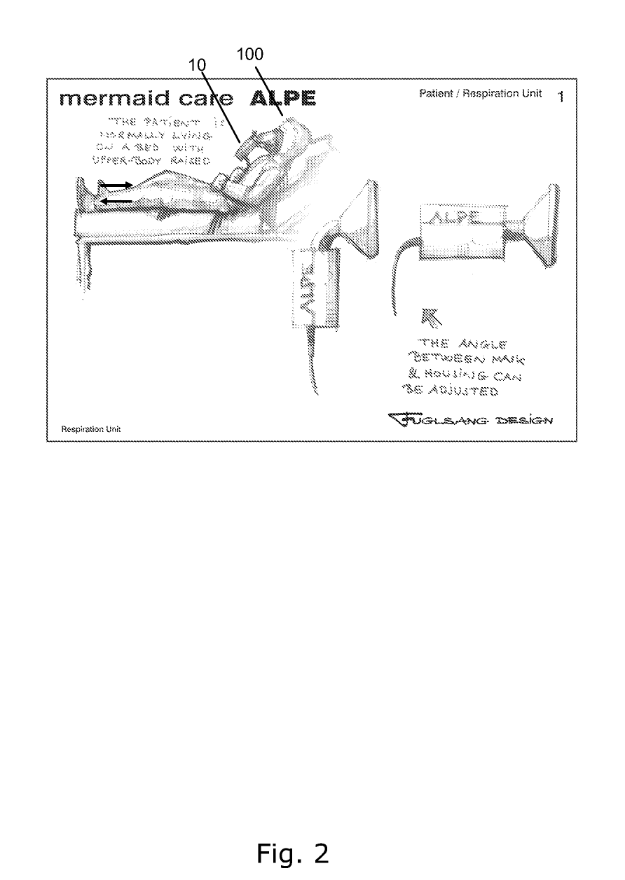

FIG. 2 is a sketch of how an individual 100 can wear a respiration mask including the gas mixing device 10 according to the present invention. It is to be noted that the relative smallness or compactness of the gas mixing device 10 allows for integration of the device 10 into an easy-to-wear respiration mask “ALPE”, which also facilitates simple and convenient wiring and gas supply to the mask in a clinical situation.

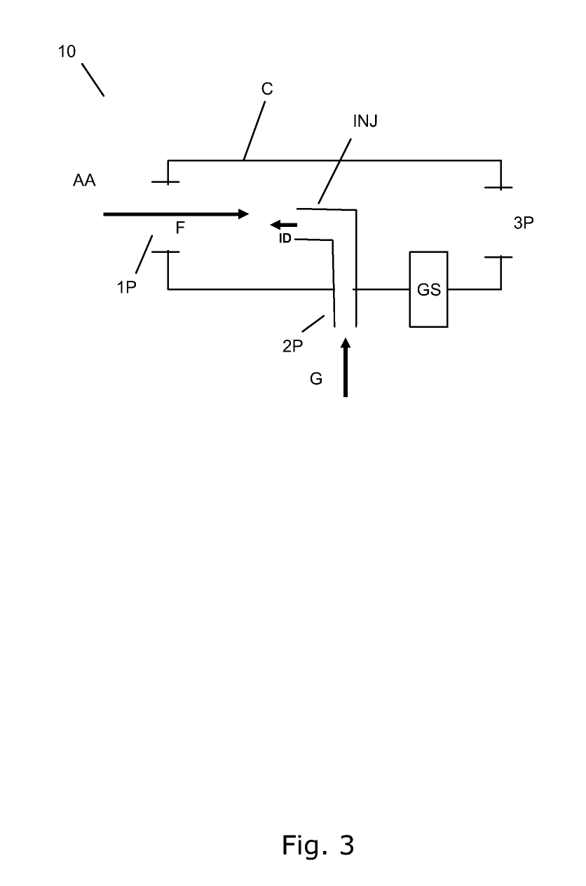

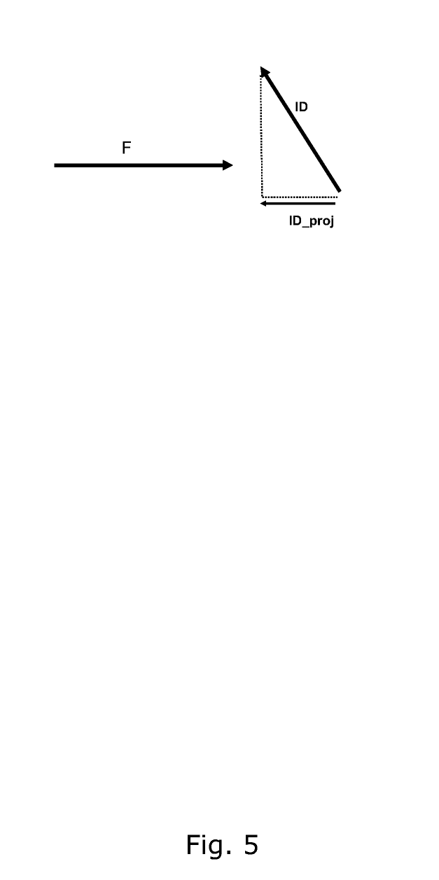

FIG. 3 is schematic drawing of a gas mixing device 10 according to the present invention. The gas mixing device 10 is intended and suitable for an air-way management system, i.e. a respiration system. The device 10 comprises an elongated chamber C, where gas mixing can takes place, i.e. the chamber should be substantially airtight or hermetically sealed except where air or gas are to flow in and/or out of the chamber C. In particular, the chamber has a first gas inlet port 1P which is arranged for intake of atmospheric air AA, either directly or through a suitable grating or filter, into the chamber C as indicated by the in-flux arrow F. The first inlet port 1P is positioned at an end section of the internal chamber. Additionally, there is provided a second gas inlet port 2P, which is arranged for intake of a gas G into the chamber C. The second gas inlet port 2P comprises an injector INJ from which the gas G can exit into the chamber C with an injection direction ID. This injection direction ID has a projection ID_proj (cf. FIG. 5), which is oppositely directed relative to the in-flow direction F of the first gas inlet port 1P so as to provide mixing of the atmospheric air AA and the gas G. Thus, by providing an up-stream direction of the incoming gas G sufficient mixing with the in-flowing air AA can be obtained.

The device 10 further has a breathing port 3P that allows an individual 100 to breathe through the gas mixing device 10. The breathing port is positioned opposite the first gas inlet port 1P within the chamber C in order to create directional flow F through the elongated chamber C. The internal diameter of the chamber C is typically 10-30 mm. The term “elongated” is, in connection with the present invention, to be understood in the broad sense that a transverse dimension of the device 10 is smaller than a longitudinal dimension of the device 10. Preferably, the transverse dimension is 1.5, 2, 3, 4, or 5 times smaller than a longitudinal dimension of the devices.

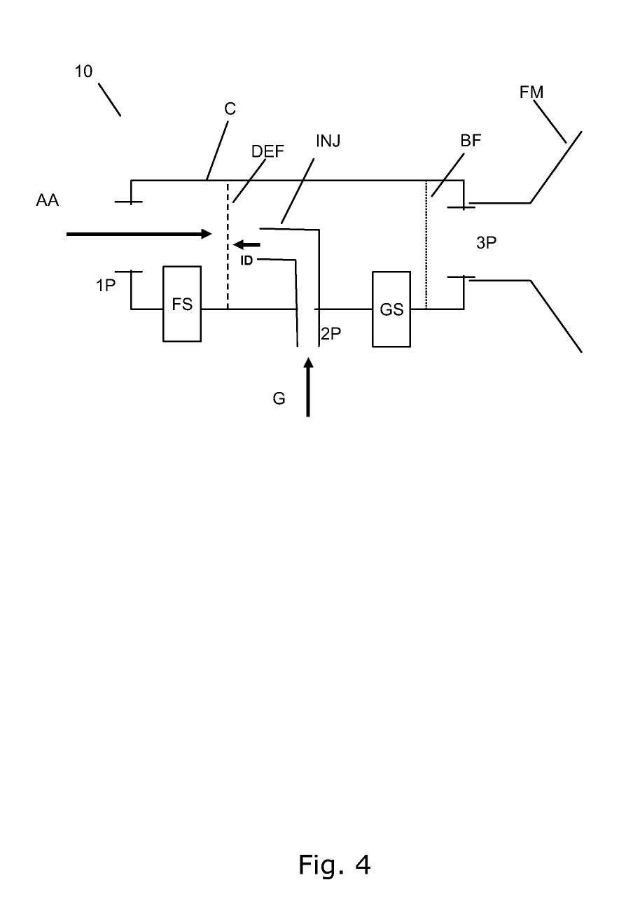

FIG. 4 is a more detailed embodiment of a gas mixing device 10 according to the present invention as compared to FIG. 3. The device 10 in particular further comprises a deflector membrane DEF positioned between the first inlet port 1P and the injector INJ. Tests and experiments performed by the present applicant has shown that carefully designed and positioned the air-permeable deflector DEF can have the function of efficiently scattering the gas G coming from injector INJ with the injection direction ID and thereby provide through mixing with in-flowing atmospheric AA on a relatively short length of the device 10.

More specifically, the deflector membrane DEF and the exit of the injector INJ is separated at least 2 mm, preferably at least 4, or more preferably at least 6 mm depending on the rate of in-flow F, the desired gas mixture, the rate of the gas G into the second port 2P among other factors. It is contemplated that the deflector DEF and the injector INJ can have an adjustable relative distance in order to dynamically vary the resulting gas mixing. The deflector DEF also has the function of separating the flow sensor FS and the gas sensor GS in order to ensure that both sensors measure independently of each other.

In FIG. 4, additionally a gas sensor GS is arranged for measuring a gas property resulting from the mixing of the atmospheric air AA and the gas G. The gas property can be any appropriate property relevant for respiratory diagnosis and/or treatment, but typically the gas sensor GS comprises an oxygen sensor. Gas composition is especially relevant, and in particular the following parameters can be measured by the gas sensor GS:
FIO2 Fraction of oxygen in inspired gas,
PIO2 Pressure of oxygen in inspired gas,
FĒCO2 Fraction of carbon dioxide in the mixed expired gas,
FE′O2 Fraction of oxygen in expired gas at the end of expiration,
FĒO2 Fraction of oxygen in the mixed expired gas,
PĒCO2 Pressure of oxygen in the mixed expired gas, and/or
PE′O2 Pressure of oxygen in expired gas at the end of expiration.

The above list is non-exhaustive as the skilled person can readily arrive at additional gas parameters or properties that can be measured, directly or indirectly, by the gas sensor GS.

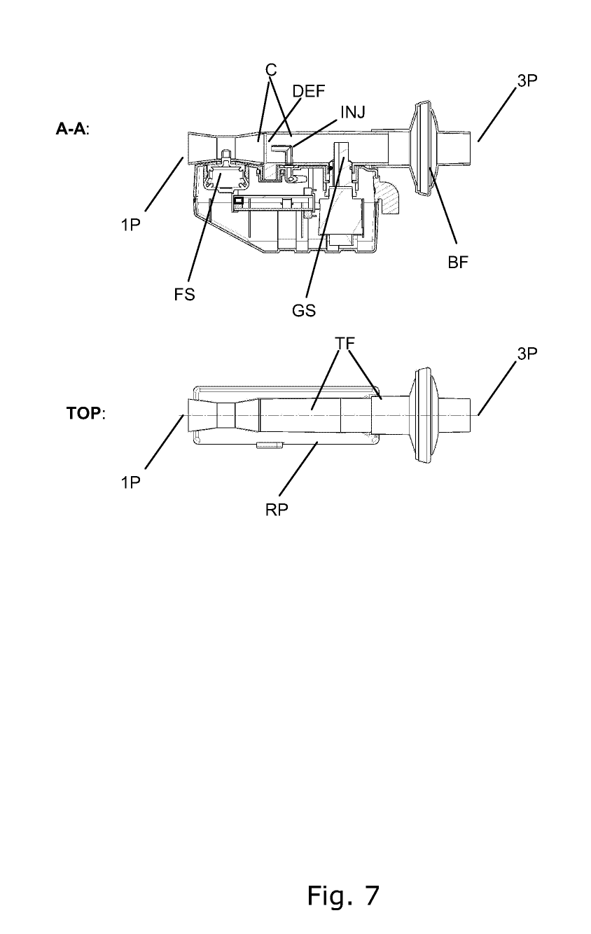

Additionally, the device 10 shown in FIG. 4 comprises a gas flow sensor FS, preferably a bi-directional flow sensor. The flow sensor FS is situated nearby the first port 1P, and preferably the gas flow sensor FS can be combined with a Venturi-contraction as will be explained and shown in connection with FIG. 7 below.

The device 10 also comprises an air-way filter BF, which is positioned between the injector INJ and the breathing port 3P. The filter can be effective against bacteria and/or vira, and possibly other contaminations. The filter BF protects the gas sensor GS, the injector INJ, and the deflector membrane DEF.

The air-way filter BF is preferably integrally formed with the chamber C so that the filter BF is non-detachable, intentionally or non-intentionally, at least by a user or an administrator of the device 10. This can be assured for example by welding, gluing or ultrasonic joining the chamber C together with the filter BF in a locking position.

As indicated in FIG. 4, the breathing port 3P is adapted to receive a face mask FM or a mouth piece (not shown). The mask FM or mouth piece can be adapted to the kind of individual, e.g. adult or child. For the case of a mouth piece, a nasal clamp should also be applied to secure air tightness. Possible the device 10 can be connected to an endotracheal tube or other kinds of respiratory tubes.

FIG. 5 is a more detailed drawing of how the inflow F trough the gas mixing device 10 is orientated relative to the injection direction ID. The injection direction ID is not anti-parallel to the flow direction F, but the injection direction has a projection ID_proj which is oppositely directed relative to the in-flow direction (F) of the first gas inlet port (1P) through the device 10. Preferably, the projection ID_proj is substantially equal to the injection direction vector ID, i.e. the injection direction ID is substantially anti-parallel to the flow direction F to provide optimum mixing but other variations and designs are possible as long as the projection ID_proj against the in-flow direction F is non-zero, i.e. the injection direction ID has an up-stream component.

Preferably, the injector INJ is dimensioned to create a laminar flow of the gas G streaming out. For the relevant gas flow rates for respiratory measurements, the applicant has found that a final part of the injector INJ should have a length-to-diameter ratio of at least 3, preferably at least 4, or more preferably at least 5. Typically, the injector INJ exit diameter is maximum 3 mm, preferably maximum 1.5 mm, or more preferably maximum 0.5 mm for most applications. Thus, the diameter is in the interval from 0.2-3 mm.

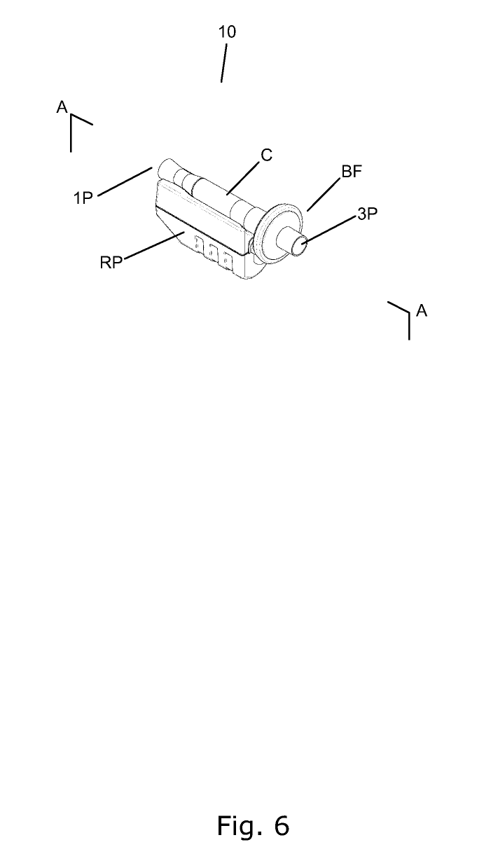

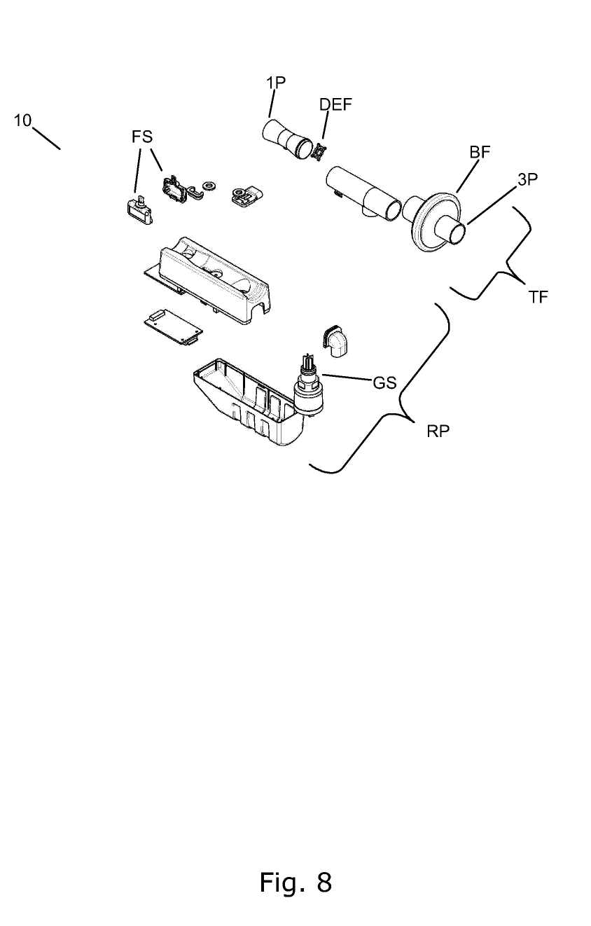

FIG. 6 is a perspective view of an embodiment of the gas mixing device 10 according to the present invention. The first inlet port 1P forms part of a Venturi-contraction around the gas flow sensor FS (not shown here). The breathing port 3P is positioned next to the filter BF, which have a diameter larger than a transverse dimension of the circular-shape chamber C in order to reduce the flow resistance through the filter BF. As explained above, the filter BF forms an integral and non-detachable part of the chamber C. This corresponds to at least part of a tube fitting TF, cf. FIG. 8. The upper part of the device 10, i.e. at least the visible part of the chamber 10 and the first 1P and third 3P inlet ports constitute a tube fitting TF, cf. FIG. 8. The tube fitting TF also comprises the second inlet port 2P comprising the injector INJ, and the tube fitting TF is adapted to receive the gas sensor GS, and the flow sensor FS. The tube fitting TF is further adapted to be received in a lower receiving part RP.

FIG. 7, AA and TOP, are cross-sectional and top views, respectively, of the gas mixing device 10 shown in FIG. 6. The cross-sectional view AA is taken along A-A indicated in FIG. 6. In the A-A view, the interior parts of the device 10, in particular within the chamber C, can be seen. In the top view TOP, the elongated cylinder-like shape of the tube fitting TF placed on top on the substantially rectangular-shaped receiving part RP can be appreciated.

FIG. 8 is an exploded view of the of the gas mixing device also shown in FIGS. 6 and 7. It should be noted that the tubular fitting TF comprises several distinct parts that can be assembled either directly after manufacturing, directly before application or at an intermediate time depending on the specific circumstances. The tubular fitting TF is preferably a single-use fitting designed only for one patient due to hygienic and safety requirements. Possibly, only the filter BF is replaced after single use. In other embodiments, the filter BF is integrally formed with the fitting TF, preferably non-detachably assembled with the fitting TF, in order to prevent re-use of the filter for safety reasons.

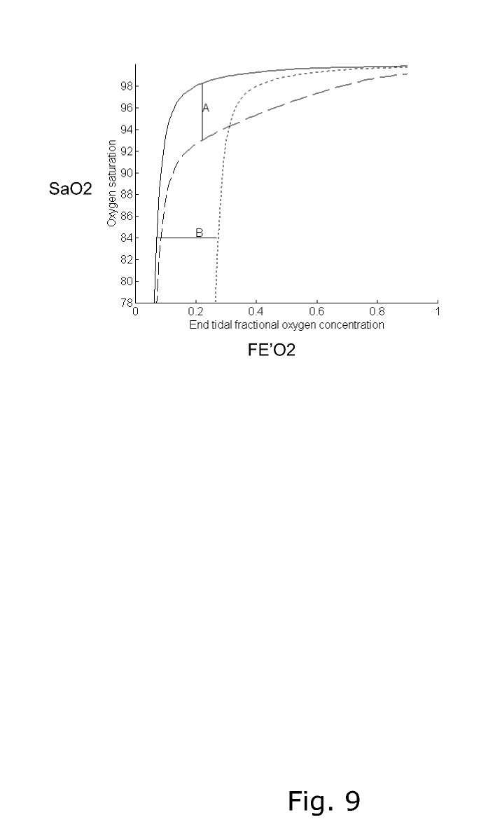

FIG. 9 is a graph showing oxygen saturation (SaO2) versus fraction of end tidal oxygen (FE′O2) that can be beneficially measured using an air-way management system according to the present invention. In addition to the inspired and/or expired gas parameters measured by gas sensor GS, it may required to measure for instance arterial oxygen saturation (SaO2) via e.g. a pulse oxymeter (SpO2). Measurements of arterial or venous blood gas samples may be taken or may be monitored continuously by invasive means and combined with measurement made according to the present invention.

FIG. 9 shows plots of the end tidal oxygen fraction (FE′O2, x-axis) against model predicted arterial oxygen saturation (SaO2, SpO2, y-axis) for 1) a normal individual with shunt=5% and Rdiff=0 kPa/(l/min) (solid line), 2) a hypothetical patient with a Rdiff or ventilation/perfusion disorder (dotted line), and 3) a hypothetical patient with a shunt disorder (dashed line). Line A illustrates the vertical displacement of the curve (V-shift) due to a shunt disorder, whilst line B illustrates the horizontal displacement of the curve (H-shift) due to a ventilation perfusion of oxygen diffusion abnormality.

In particular, the following non-exhaustive of list respiratory parameters may be determined:
Vt Tidal volume, i.e. volume of gas breathed per breath.
f Respiratory frequency, i.e. number of breaths per minute.
VO2 Oxygen consumption, i.e. the amount of oxygen consumed by the tissues per minute.
Vd Dead space i.e. the volume of the lung not involved in exchanging gases with the blood.
shunt Respiratory parameter representing the faction of blood not involved in gas exchange.
Rdiff Respiratory parameter representing a resistance to oxygen diffusion across the alveolar lung capillary membrane.
\dot{V} Ventilation.
\dot{V}/\dot{Q} Respiratory parameter representing the balance between ventilation and perfusion in a region of the lung.
V-shift Respiratory parameter representing a vertical shift in plots of FIO2 against SaO2, FIO2 against SpO2, FE′O2 against SaO2, or FE′O2 against SpO2 .
H-shift Respiratory parameter representing a horizontal shift in plots of FIO2 against SaO2, FIO2 against SpO2, FE′O2 against SaO2, or FE′O2 against SpO2.

For additional reference on these respiratory parameters, the reader is referred to WO 00/45702 regarding the ALPE-device and method, which is hereby by included by reference in its entirety.

One of the key features of the control unit CU is to control the mixing of a given air blend in real time. To that end, a regulation loop based on a conventional proportional-integral-derivative (PID) control system with negative feedback which opens a flow valve (not shown) in proportion to the current needs. This type of control system is based on the difference between the error between the required level and the measured level and it makes it possible to make a fast regulation with small error. Some parameters may need to be adjusted in order to obtain a stable system without oscillation and with acceptable step response with none or with an acceptable overshoot.

Depending on tube fitting TF resistance, valve reaction times, valve hysteresis and flow measurement delay these parameters may not be known. This makes it difficult to calculate the final parameter values. So the approach to this issue is to design a complete control system where a few regulation parameters are to be defined. These loop parameters will be then defined after the air-way management system is put together and a few measurements have been made.

The PID regulation parameters Kₚ, Kᵢ and K_{d} are tuned by using the Ziegler-Nichols Method. In case that these values tend to oscillate, the more conservative Tyreus and Luyben tuning will be used. These values damp the control in order to reduce oscillatory effects in the control system. The loop will be designed so the loop is stable even with variations of sensors FS and GS and other component affecting regulation loop. This will be achieved by having margin to the point of oscillation.

The oxygen PID controller regulation is implemented due to the real-time requirement and the reliability requirement. A brief outline of some relevant gas control equations are given below.

$$When desired FIO 2 \text{>} 0.21 ( Gas G = Oxygen )$$

$${FIO}_{2} = \frac{{Q}_{air} \cdot 0 , 21 + {Q}_{gas}}{{Q}_{air} + {Q}_{gas}}$$

$${Q}_{gas} = {Q}_{air} \cdot k$$

$${FIO}_{2} = \frac{0 , 21 + k}{1 + k}$$

$$and$$

$$k = \frac{0 , 21 - {FIO}_{2}}{{FIO}_{2} - 1}$$

In order to achieve a FIO2 level from 0.22-0.40 it requires a k in the range 0.01-0.32.

$$When desired FIO 2 \text{<} 0.21 ( Gas G = Nitrogen )$$

$${FIO}_{2} = \frac{{Q}_{air} \cdot 0 , 21}{{Q}_{air} + {Q}_{gas}}$$

$${Q}_{gas} = {Q}_{air} \cdot k$$

$${FIO}_{2} = \frac{0 , 21}{1 + k}$$

$$and$$

$$k = \frac{0 , 21 - {FIO}_{2}}{{FIO}_{2}}$$

In order to achieve a FIO2 level from 0.15-0.20 it requires a k in the range 0.4-0.01.

Although the present invention has been described in connection with the specified embodiments, it is not intended to be limited to the specific form set forth herein. Rather, the scope of the present invention is limited only by the accompanying claims. In the claims, the term “comprising” does not exclude the presence of other elements or steps. Additionally, although individual features may be included in different claims, these may possibly be advantageously combined, and the inclusion in different claims does not imply that a combination of features is not feasible and/or advantageous. In addition, singular references do not exclude a plurality. Thus, references to “a”, “an”, “first”, “second” etc. do not preclude a plurality. Furthermore, reference signs in the claims shall not be construed as limiting the scope.

1. A gas mixing device (10) for an air-way management system, the device comprising:
a) an elongated chamber (C),
b) a first gas inlet port (1P) being arranged for intake of atmospheric air (AA) into the chamber, the first inlet port being positioned at an end section of the internal chamber,
c) a second gas inlet port (2P) arranged for intake of a gas (G) into the chamber (C), the second gas inlet port comprising an injector (INJ) from which the gas can exit into the chamber with an injection direction (ID), the injection direction having a projection (ID_proj) being oppositely directed relative to an in-flow direction (F) of the first gas inlet port (1P) so as to provide mixing of the atmospheric air and the gas, and
d) a breathing port (3P) for allowing an individual to breathe through the gas mixing device, the breathing port being positioned opposite the first gas inlet port (1P) within the chamber (C).

2. The device according to claim 1, wherein the device further comprises a deflector membrane (DEF) positioned between the first inlet port (1P) and the injector (INJ).

3. The device according to claim 1, where the distance between the deflector membrane (DEF) and the exit of the injector (INJ) is at least 2 mm, preferably at least 4, or more preferably at least 6 mm.

4. The device according to claim 1 or 3, where the injector exit diameter is maximum 3 mm, preferably maximum 1.5 mm, or more preferably maximum 0.5 mm.

5. The device according to claim 1, wherein the device further comprises a gas sensor (GS) arranged for measuring a gas property resulting from the mixing of the atmospheric air (AA) and the gas (G).

6. The device according to claim 5, wherein the gas sensor (GS) comprises an oxygen sensor.

7. The device according to claim 1, wherein the device further comprises a gas flow sensor (FS), preferably a bi-directional flow sensor.

8. The device according to claim 7, wherein the device comprises a Venturi-contraction where the flow sensor (FS) is positioned.

9. The device according to claim 1, wherein the device further comprises an air-way filter (BF) positioned between the injector (INJ) and the breathing port (3P).

10. The device according to claim 1, wherein the breathing port (3P) is adapted to receive a face mask (FM) or a mouth piece.

11. The device according to claim 1, wherein the total internal volume of the gas mixing device is maximum 10 cm³, preferably maximum 15 cm³, or most preferably maximum 20 cm³.

12. The device according to claim 1, wherein the breathing resistance is maximum 0.2 Pa*s/L, preferably maximum 0.4 Pa*s/L, or most preferably maximum 0.6 Pa*s/L, at a flow through the device of approximately 50 L/min.

13. A tube fitting (TF) forming part of the gas mixing device (10) according to claim 1, the tube fitting comprises at least part of the elongated chamber (C), the first inlet port (1P), the second inlet port (2P) comprising the injector (INJ) and the breathing port (3P), the tubular fitting being adapted to receive:
optionally, the gas sensor (GS), and optionally
the flow sensor (FS).

14. The tube fitting according to claim 13, the tube fitting being adapted to provide a tactile and/or audio response to a user upon correct assembly upon a receiving part (RP) of the gas mixing device (10).

15. The tube fitting according to claim 13, the tube fitting being disposable after a single use.

16. The tube fitting according to claim 15, wherein the tube fitting is arranged so as to allow for single use only.

17. A respiration mask or a mouth piece comprising a gas mixing device according to any of claims 1-12.

18. An air-way management system for measurement of one or more respiratory parameters of an individual, the system comprising:
a respiration mask or a mouth piece according to claim 17,
a gas supply for supplying the second inlet port with the gas, and
a control unit interconnected with the gas supply,
wherein the control unit is arranged to receive output signals from optionally the gas sensor (GS) and optionally the flow sensor (FS), and control the gas supply in response to said output signals.

19. Use of a gas mixing device according to any of claims 1-12 for measurement of one or more respiratory parameters of an individual.
